(19) United States
(12) Patent Application Publication
(10) Pub. No.: US 2006/0051435 A1
(43) Pub. Date: Mar. 9, 2006

(54) Nutritional supplement for body fat reduction

(76) Inventors: Ronald G. Udell, Beverly Hills, CA (US); Kenneth Israel, Pasadena, CA (US)

Correspondence Address: Scott D. Rothenberger, Esq.;DORSEY & WHITNEY LLP, Intellectual Property Department, 50 South Sixth Street, Suite 50, Minneapolis, MN, 55402-1498, US

(21) Appl. No.: 11/205,789

(22) Filed: Aug. 17, 2005

Related U.S. Application Data
(60) Provisional application No. 60/602,921, filed on Aug. 19, 2004.

(51) Int. Cl. A61K 36/48; A61K 36/185

(52) U.S. Cl. 424/725; 424/757

(57) ABSTRACT

The present invention is directed to compositions containing a conjugated linoleic acid derivative, a banaba extract and an extract of Phaseolis vulgaris and methods of treatment for weight loss.

### CROSS-REFERENCE TO RELATED APPLICATION(S)

This application claims benefit under 35 U.S.C. § 1.19(e) to U.S. Ser. No. 60/602,921, filed on Aug. 19, 2004, entitled “Nutritional Supplement for Body Fat Reduction”, by Ronald G. Udell and Kenneth Israel (US Attorney Docket No. 34625/US), the contents of which are incorporated herein in their entirety for all purposes.

### FIELD OF THE INVENTION

The present invention relates to formulations, such as soft gelatin capsules, containing a combination of conjugated linoleic acid, a starch blocker and Banaba extract that includes Corosolic acid.

### BACKGROUND OF THE INVENTION

Over the past several decades the world's population, in general, has been becoming overweight. In many instances, excessive weight gain increases the risk of diabetes, hypertension, cardiac diseases or kidney disease among other diseases or conditions related to obesity. It is not just an affliction of the elderly; in fact, many recent studies show that increasing numbers of juveniles and teenagers are developing weight related afflictions.

Today's sedentary lifestyle unfortunately helps to promote excessive weight gain in individuals. One of the problems with the Western diet during the last fifty years has been excessive consumption of linoleic acid, due to the introduction of margarine, seed oils such as corn oil and safflower oil, and the modern artificial feeding methods of cattle that have raised the linoleic acid content of meat. At the same time, the consumption of beneficial fatty acids such as omega-3 fats (fish, flax, perilla) and conjugated linoleic acid (CLA) has decreased.

There are many treatments and diet programs available to help promote a healthy lifestyle and weight loss. Most diet programs consist of reducing caloric intake, reducing the amount of carbohydrates consumed, and appropriate exercise. Surgical treatments are available and include stomach stapling, gastric by-pass and liposuction

Unfortunately, an individual will often lose weight on a diet program but then later succumb to old eating habits. Exercise often becomes a luxury due to life style constraints and the individual lapses into cycle of dieting, weight loss, cessation of the diet and weight gain, only to repeat this process over and over.

Surgical procedures can be quite expensive and inconvenient. Many procedures are not paid for by insurance companies and the individual is forced with a decision to either pay for the procedure themselves or forgo the surgery. Additionally, surgery can result in the person not being ambulatory for a week or more and can be quite painful.

Weight gain is a complex problem. The health conscious public is concerned about excessive weight gain, but does not generally want to maintain strict diets to lose or maintain a healthy weight. Weight gain is often a combination of reduced metabolism of glucose, the individual's inability to block carbohydrate absorption from the diet and/or the inability to reduce body fat. A solution to one or more of these problems is sought by many people, young and old alike

There is a need in the art for a nutritional supplement that counteracts one or more of the afore-mentioned weight related afflictions.

### BRIEF SUMMARY OF THE INVENTION

The present invention pertains to compositions for the reduction of body fat, the increase in glucose metabolism, inhibiting absorption of carbohydrates and/or weight loss. The invention also pertains to methods to accomplish one or more of these goals. The invention further pertains to packaged nutraceutical formulations to treat such conditions.

In one aspect, the present invention surprisingly provides that a combination of banaba extract, a starch blocker and a conjugated linoleic acid derivative can improve one or more of the afore-mentioned health issues. The composition can be ingested in the form of a tablet, pill, lozenge, soft gel capsule, in a health food snack, in a drink, etc. The recommended daily amount can be taken in one or more portions.

Banaba (Lagerstroemia Speciosa L.), also known as Queen's flower, pride of India, or queen's crape myrtle, belongs to the botanical family lythraceae and contains corosolic acid and other beneficial phytochemicals. Corosolic acid (2-α-hydroxyursolic acid, CAS# 52213-27-1) is a triterpenoid with a molecular weight of 743.63 daltons and is a lipophilic, polar compound that is extracted from the leaves of Lagerstroemia Speciosa L. A suitable commercial source of corosolic acid is available from Soft Gel Technologies, Inc. of Los Angeles, Calif., USA, and is known by the trademark GLUCOTRIM™ (available as a 1% or 3% corosolic acid extract).

Conjugated linoleic acid derivatives (CLA's) include any conjugated linoleic acid or octadecadienoic fatty acid, including all positional and geometric isomers of linoleic acid with two conjugated carbon-carbon double bonds at any position in the molecule. Additionally, the term includes salts and esters thereof, including triglycerides.

Starch blockers are known in the art and the term is intended to include, but is not limited to, alpha amylase inhibitors, alpha-glucoside inhibitors and glucosidase inhibitors. In particular, the present invention includes starch blockers based on derivatives from white kidney beans (Phaseolis vulgaris). Phaseolamin is a partially-purified protein extract of white kidney beans that binds to alpha-amylase enzymes, which are responsible for the digestive breakdown of starch. It has been proposed that phaseolamin inhibits the alpha-amylase breakdown of starch by non-competitively binding the enzyme to prevent the hydrolysis of the alpha-1,4-glycosidic linkages in the starch molecule.

The composition can include additional additives, such as antioxidants, stabilizers, fillers, carriers, etc.

Suitable optional carriers for the ingredients include, for example, wheat germ oil, rice bran oil, or yellow beeswax.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive.

### DETAILED DESCRIPTION

The present invention pertains to the surprising discovery that compositions that include a banaba extract, a conjugated linoleic acid derivative and a starch blocker are effective in the treatment for reduction of body fat, increasing glucose metabolism, inhibiting absorption of carbohydrates and/or weight loss in individuals in need thereof.

The composition of the invention can be incorporated into various foods, drinks, snacks, etc. In one aspect, the composition can be sprinkled onto a food product, prior to consumption. If sprinkled onto a food product, a suitable carrier such as starch, sucrose or lactose, can be used to help distribute the concentration of the active ingredients, making it easier to apply to the food product.

The compositions of the present invention can also be provided as supplements in various prepared food products For the purposes of this application, prepared food product means any natural, processed, diet or non-diet food product to which a composition of the invention has been added. The compositions of the present invention can be directly incorporated into many prepared diet food products, including, but not limited to diet drinks, diet bars and prepared frozen meals. Furthermore, the compositions of the inventions can be incorporated into many prepared non-diet products, including, but not limited to candy, snack products such as chips, prepared meat products, milk, cheese, yogurt, sport bars, sport drinks, mayonnaise, salad dressing, bread and any other fat or oil containing foods. As used herein, the term “food product” refers to any substance fit for human or animal consumption.

The compositions of the invention can be added to various drinks, such as fruit juices, milkshakes, milk, etc.

The compositions of the invention are intended not only for weight loss, a method to increase glucose metabolism, a method to reduced body fat, and/or block carbohydrate absorption from the diet, but as nutritional supplements to maintain the results obtained from use of the composition(s). This is a life style change, wherein carbohydrate intake is diminished such that one or more of the aforementioned goals is achieved. That is, once the individual achieves the result(s) desired, the use of the compositions of the invention to maintain that achievement is just as important and first reaching the goal. Therefore, the compositions of the invention can be viewed as supplements to help an individual prevent one or more of the afore-mentioned conditions from reoccurring.

The preferred method of administration is oral. The compositions of the invention can be formulated with suitable carriers such as starch, sucrose or lactose in tablets, capsules, solutions, syrups and emulsions. The tablet or capsule of the present invention may be coated with an enteric coating that dissolves at a pH of about 6.0 to 7.0. A suitable enteric coating, which dissolves in the small intestine but not in the stomach, is cellulose acetate phthalate.

The term “banaba extract” is recognized in the art and is intended to include the extraction product from banaba (Lagerstroemia Speciosa L.), also known as Queen's flower, pride of India, or queen's crape myrtle, and contains corosolic acid and other phytochemicals. In one aspect, the corosolic acid content of the extract is about 3% by weight of the dried material. In another aspect, the corosolic acid content of the extract is about 1% by weight of the dried material. In still yet another aspect, the corosolic acid content of the extract is between about 1% and about 3% by weight of the dried material.

Generally, between about 5 milligrams and about 60 milligrams of corosolic acid can be included in a composition of the invention, in particular, between about 8 milligrams and about 56 milligrams, and more particularly between about 10 milligrams and about 50 milligrams on a weight basis.

Typically a composition is provided that includes about 8 milligrams of the corosolic acid or banaba extract. Generally, two, three, four or more dosages of the composition are taken over the course of a day to provide between about 8 and about 56 milligrams of corosolic acid, e.g., between about 24 and about 48 mg per day.

The term “conjugated linoleic acid” is intended to include any conjugated linoleic acid or octadecadienoic fatty acid, including all positional and geometric isomers of linoleic acid with two conjugated carbon-carbon double bonds at any position in the molecule. Suitable examples of CLA include cis- and trans isomers (“E/Z isomers”) of the following positional isomers: 2,4-octadecadienoic acid, 4,6-octadecadienoic acid, 6,8-octadecadienoic acid, 7,9-octadecadienoic acid, 8,10-octadecadienoic acid, 9,11-octadecadienoic acid, 10,12 octadecadienoic acid and 11,13 octadecadienoic acid. As used herein, “CLA” encompasses a single isomer, a selected mixture of two or more isomers, and a non-selected mixture of isomers obtained from natural sources, as well as synthetic and semisynthetic CLA. The term is intended to include non-naturally occurring isomers of CLA.

CLA is an omega 6 oil. Suitable sources of CLA include, for example, sunflower oil, corn oil, or safflower oil. Typically, the oils provide a CLA content of between about 70 and about 90% (by weight), more particularly between about 75 and about 85%, and even more particularly, between about 78 and about 84% by weight.

It is believed that CLA reduces body fat by enhancing insulin sensitivity so that fatty acids and glucose can pass through muscle cell membranes and away from fat tissue. This results in an improved muscle to fat ratio. Compelling evidence indicates that CLA can promote youthful metabolic function and reduce body fat.

The term “isomerized conjugated linoleic acid” refers to a CLA synthesized by chemical methods (e.g., aqueous alkali isomerization, non-aqueous alkali isomerization, or alkali alcoholate isomerization).

The term “conjugated linoleic acid derivative” refers to any compound or plurality of compounds containing conjugated linoleic acids or derivatives thereof. Examples include fatty acids, alkyl esters, triglycerides of conjugated linoleic acid as well as nutritionally acceptable salts thereof.

It should be understood that “triglycerides” of CLA contain CLA at any or all of three positions (e.g., SN-1, SN-2, or SN-3 positions) on the triglyceride backbone. Accordingly, a triglyceride containing CLA can contain any of the positional and geometric isomers of CLA.

“Esters” of CLA include any and all positional and geometric isomers of CLA bound between the carboxylic acid portion to an alcohol or any other chemical group, including, but not limited to physiologically acceptable, naturally occurring alcohols (e.g., methanol, ethanol, propanol). Therefore, an ester of CLA or esterified CLA may contain any of the positional and geometric isomers of CLA.

The phrase “non-naturally occurring isomers” of CLA includes, but is not limited to c11,t13; t11,c13; t11,t13; c11,c13; c8,t10; t8,c10; t8,t10; c8,c10; and trans-trans isomers of octadecadienoic acid, and does not include t10,c12 and c9,t11 isomers of octadecadienoic acid. “Non-naturally occurring isomers” may also be referred to as “minor isomers” of CLA as these isomers are generally produced in low amounts when CLA is synthesized by alkali isomerization.

The term, “low impurity” CLA refers to CLA compositions, including free fatty acids, alkylesters, and triglycerides, which contain less than 1% total 8,10 octadecadienoic acids, 11,13 octadecadienoic acids, and trans-trans octadecadienoic acids.

The abbreviation, “c” encompasses a chemical bond in the cis orientation, and “t” refers to a chemical bond in the trans orientation. If a positional isomer of CLA is designated without a “c” or a “t”, then that designation includes all four possible isomers. For example, 10,12 octadecadienoic acid encompasses c10,t12; t10,c12; t10,t12; and c10,c12 octadecadienoic acid, while t10,c12 octadecadienoic acid or CLA refers to just the single isomer.

Salts of CLA include salts suitable for pharmaceutical/nutraceutical uses (“pharmaceutically-acceptable salts”), salts suitable for veterinary uses, etc. Such salts may be derived from acids or bases, as is well-known in the art.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4 chlorobenzenesulfonic acid, 2-naphthalenesulfonic acid, 4-toluenesulfonic acid, cycloalkylsulfonic acids (e.g., camphorsulfonic acid), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion), an ammonium ion or coordinates with an organic base (e.g., ethanolamine, diethanolamine, triethanolamine, N-methylglucamine, morpholine, piperidine, dimethylamine, diethylamine, etc.).

A suitable CLA for preparation of the compositions of the invention, is known as TONALIN®, and is available from Cognis Nutrition & Health, LaGrange, Ill., USA.

Generally, between about 250 milligrams and about 500 milligrams of CLA can be included in a composition of the invention, in particular, between about 250 milligrams and about 400 milligrams, and more particularly between about 300 milligrams and about 350 milligrams on a weight basis.

Typically a composition is provided that includes between about 250 and about 500 milligrams of the CLA. Generally, two, three, four or more dosages of the composition are taken over the course of a day to provide between about 500 and about 4000 milligrams of CLA, e.g., about 2000 milligrams per day.

Starch blockers are known in the art and the term is intended to include, but is not limited to, alpha amylase inhibitors, alpha-glucoside inhibitors and glucosidase inhibitors. In particular, the present invention includes starch blockers based on derivatives from white kidney beans (Phaseolis vulgaris). Phaseolamin is a partially-purified protein extract of white kidney beans that binds to alpha-amylase enzymes, which are responsible for the digestive breakdown of starch. It has been proposed that phaseolamin inhibits the alpha-amylase breakdown of starch by non-competitively binding the enzyme to prevent the hydrolysis of the alpha-1,4-glycosidic linkages in the starch molecule.

Alpha-glucosidase is an enzyme that breaks disaccharides into their respective monosaccharide units. Alpha-glucosidase inhibitors prevent the enzyme from performing this function. A wide variety of alpha-glucosidase inhibitors are known and any suitable inhibitor can be used in the compositions and methods of the present invention. Examples of suitable alpha-glucosidase inhibitors include, but are not limited to, voglibose (see U.S. Pat. No. 6,200,958 to Odaka et al.), acarbose (see U.S. Pat. No. 5,643,874 to Bremer et al.), and touchi extract. Touchi is a traditional Chinese food derived from soybeans. Touchi is prepared by first steaming and then fermenting soybeans with Aspergillus species bacteria

Alpha-amylase is an enzyme that functions to break the alpha-1,4-glycosidic linkages present in starch. This breaks the complex starch molecule into smaller units, such as disaccharides, that can be further digested by other enzymes, such as alpha-glucosidase. Alpha-amylase inhibitors prevent the enzyme from hydrolyzing the alpha-1,4-glycosidic bond, and therefore prevent the breakdown of starch. A wide variety of alpha-amylase inhibitors are known, and any suitable inhibitor can be used in the compositions and methods of the present invention. Examples of suitable alpha-amylase inhibitors include, but are not limited to, an inhibitor extracted from wheat (see U.S. Pat. No. 3,950,319 to Schmidt et al.), Amylostatin-A (see U.S. Pat. No. 4,010,258 to Murao), and phaseolamin.

In one aspect, the compositions of the invention include the alpha-amylase inhibitor phaseolamin. Phaseolamin is an extract of the white kidney bean (Phaseolus vulgaris). The extract is water-soluble and rich in protein content. Phaseolamin is readily available from numerous commercial suppliers. Phaseolamin PHASEOLAMIN 2250®, available from Pharmachem Laboratories of Kearny, N.J. and also known as PHASE 2®, is a standardized extract particularly well-suited for inclusion in the compositions according to the present invention. This phaseolamin demonstrates a high ability to block alpha-amylase activity.

Generally, between about 125 milligrams and about 350 milligrams of starch blocker can be included in a composition of the invention, in particular, between about 125 milligrams and about 333 milligrams, and more particularly between about 150 milligrams and about 250 milligrams on a weight basis.

Typically a composition is provided that includes between about 125 and about 333 milligrams of the starch blocker. Generally, two, three, four or more dosages of the composition are taken over the course of a day to provide between about 250 and about 3000 milligrams of the starch blocker, e.g., between about 1000 milligrams and about 2000 milligrams.

Formulation of the compositions of the invention into a soft gel capsule can be accomplished by many methods known in the art. Often the formulation will include an acceptable carrier, such as an oil, or other suspending or emulsifying agent. However, use of CLA eliminates the need for a carrier, as the CLA acts as a carrier. This provides a quantifiable amount of CLA with a nutritional benefit. Additionally, this increases the efficiency of preparation and is more economical than formulations that require an additional carrier.

Suitable optional carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, wheat germ oil, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The formulations of the invention are considered dietary supplements useful to treat the weight related afflictions identified herein in individuals in need thereof.

Alternatively, the formulations of the invention are also considered to be nutraceuticals. The term “nutraceutical” is recognized in the art and is intended to describe specific chemical compounds found in foods that may prevent disease or ameliorate an undesirable condition. Banaba extract (corosolic acid), conjugated linoleic acid derivatives and starch blockers are such compounds.

The formulations of the invention can further include various ingredients to help stabilize, or help promote the bioavailability of the components of the beneficial compositions of the invention or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, hyaluronic acid, phospholipids, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

Generally, between about 50 milligrams and about 250 milligrams of a phospholipid can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of a phospholipid. Generally, two, three, four or more dosages of the composition are taken over the course of a day to provide between about 50 milligrams and about 1000 milligrams of phospholipid, e.g., between about 250 milligrams and about 500 milligrams.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, camosic acid and rosmanol found in rosemary and hawthorn extract, proanthocyanidins such as those found in grapeseed or pine bark extract, and green tea extract.

Generally, between about 50 milligrams and about 250 milligrams of an antioxidant(s) can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of an antioxidant. Generally, two, three, four or more dosages of the composition are taken over the course of a day to provide between about 50 milligrams and about 1000 milligrams of antioxidant(s), e.g., between about 250 milligrams and about 500 milligrams.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC) (See below).

The compositions of the invention can further include additives that are beneficial in the treatment of weight loss, increase glucose metabolism, reduce body fat, and/or block carbohydrate absorption from the diet. Such additives can include, for example, pyruvate, i.e., calcium pyruvate, Gynmema Sylvestris, green tea, polynicotinate, i.e., chromium polynicotinate, bitter orange, yerba mate, glucomannan, coleus forskoli, jojoba, guggul lipds, NOPI (Phosphalean), ephedra, yohimbe, citrus aurantium coffee (caffeine), chromium picolinate, garcinia cambodgia, Caralluma Fimbriata extract, fenugreek and its derivatives, L-carnitine as well as its salts and esters, ginseng, chocolate extracts containing phenyl ethyl amine and/or theobromine, tannins, polyphenols, coffee extracts (including green coffee) such as chlorogenic acids, cinnamon, Hoodia Gordonii, lotus seed or root and combinations of all of the above.

Pyruvate is believed to accelerate fat loss by increasing mitochondrial activity. Pyruvate is a carbohydrate naturally found in red apples, cheeses, and red wine. Pyruvic acid is a carboxylic acid; therefore, suitable carboxylic acid salts and esters can be used as an additive. These include calcium and sodium salts of pyruvic acid.

Generally, between about 50 milligrams and about 250 milligrams of a pyruvic acid can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of pyruvic acid. Generally, two, three, four or more dosages of the composition are taken over the course of a day to provide between about 50 milligrams and about 1000 milligrams of pyruvic acid, e.g., between about 250 milligrams and about 500 milligrams.

Gymnema Sylvestris is a known anti-diabetic agent. It helps to balance blood sugar and decreases sugar cravings in individuals. The hypoglycemic (blood sugar-lowering) action of gymnema leaves has been documented for over 80 years. The blood sugar-lowering action is gradual and differs from the rapid effect of many prescription hypoglycemic drugs.

Gymnema leaves raise insulin levels in individuals by regeneration of the cells in the pancreas that secrete insulin. Gymnema also improves uptake of glucose into cells by increasing the activity of glucose utilizing enzymes, and prevents adrenaline from stimulating the liver to produce glucose, thereby reducing blood sugar levels. The leaves are also noted for lowering serum cholesterol and triglycerides.

Gymnema Sylvestris leaf extract, notably the peptide Gurmarin component, has been found to interfere with the ability of the taste buds on the tongue to taste sweet and bitter. Gymnemic acid has a similar effect. The leaf extracts contain gymnemic acid which inhibits hyperglycemia and also acts as a cardiovascular stimulant.

Generally, between about 50 milligrams and about 250 milligrams of gymnema sylvestris, an extract thereof, or an isolated component thereof can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of gymnema sylvestris, an extract thereof, or an isolated component thereof. Generally, two, three, four or more dosages of the composition are taken over the course of a day to provide between about 50 milligrams and about 1000 milligrams of gymnema sylvestris, an extract thereof, or an isolated component thereof, e.g., between about 250 milligrams and about 500 milligrams.

Green tea is known to accelerate calorie burning via increased thermogenesis. Green tea contains a number of polyphenolic compounds. The catechin epigallocatechin gallate (EGCG) is the most abundant with greater than 50% of total tea catechins. It is also believed to be the most pharmacologically active. The other main catechins are epicatechin (EC), epicatechin gallate (ECG), and epigallocatechin (EGC).

Generally, between about 50 milligrams and about 250 milligrams of green tea or the polyphenolic compound(s) can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of the green tea or polyphenolic compound(s). Generally, two, three, four or more dosages of the composition are taken over the course of a day to provide between about 50 milligrams and about 1000 milligrams of the green tea, e.g., between about 250 milligrams and about 500 milligrams.

Polynicotinates are salts of nicotinic acid. Chromium polynicotinate, in particular, is a trace mineral that helps regulate carbohydrate metabolism. Since all carbohydrates are reduced in the body into simple glucose, chromium polynicotinate provides the go-between action by “plugging” serum glucose from the bloodstream directly to the muscle cell. Chromium is a necessary component for carbohydrate metabolism, glucose regulation, and energy production.

Chromium polynicotinate is a mineral utilized in the regulation of blood sugar. It is involved in the metabolism of glucose and is a key component for energy. The ability to maintain stable blood sugar levels is often jeopardized by diets that are often high in white flour, refined sugar and junk food. Chromium polynicotinate facilitates and/or stimulates the metabolism of sugar, fat and cholesterol in the body, as well as the function of insulin.

Generally, between about 50 milligrams and about 250 milligrams of a polynicotinate can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of polynicotinate. Generally, two, three, four or more dosages of the composition are taken over the course of a day to provide between about 50 milligrams and about 1000 milligrams of polynicotinate, e.g., between about 250 milligrams and about 500 milligrams.

Bitter orange (citrus aurantium) is a known fat burner. Bitter orange helps to increase the metabolic rate at which calories and fat are burned. Synephrine is the primary active alkaloid in Bitter orange. Synephrine stimulates the adrenal gland to effect fat burning, appetite suppression and natural energy.

Generally, between about 50 milligrams and about 250 milligrams of a bitter orange can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of bitter orange. Generally, two, three, four or more dosages of the composition are taken over the course of a day to provide between about 50 milligrams and about 1000 milligrams of bitter orange, e.g., between about 250 milligrams and about 500 milligrams.

Yerba mate is known to help oxidize body fat. The oxidation greatly enhances the rate at which fat will be broken down and burned away. Mateine is the primary alkaloid in Yerba mate. Mateine is a close relative to natural caffeine without any of the negative side effects. Meteine immediately and smoothly enhances energy levels, and suppresses an individual's appetite while avoiding any jitteriness, nervousness or stomach aches.

Generally, between about 50 milligrams and about 250 milligrams of a yerba mate can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of yerba mate. Generally, two, three, four or more dosages of the composition are taken over the course of a day to provide between about 50 milligrams and about 1000 milligrams of yerba mate, e.g., between about 250 milligrams and about 500 milligrams.

Glucomannan is obtained from the roots of the Konjac Plant and aids in fat loss. It is believed that glucomannan prevents fats from entering the bloodstream while the individual's appetite is suppressed.

Generally, between about 50 milligrams and about 250 milligrams of a glucomannan can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of glucomannan. Generally, two, three, four or more dosages of the composition are taken over the course of a day to provide between about 50 milligrams and about 1000 milligrams of glucomannan, e.g., between about 250 milligrams and about 500 milligrams.

Another additive suitable for co-administration with the compositions of the invention is Coleus Forskoli. This herb has an active ingredient in it called forskolin. Forskolin is a diterpene that activates adenylate cyclase and raises cyclic AMP levels in a variety of tissues. Cyclic AMP is an important cell regulating compound. cAMP is formed when a stimulatory hormone (e.g., epinephrine) binds to a receptor site on the cell membrane and stimulates the activation of adenylate cyclase. This enzyme is incorporated into all cellular membranes and only the specificity of the receptor determines which hormone will activate it in a particular cell. Forskolin appears to bypass this need for direct hormonal activation of adenylate cyclase. As a result of this direct activation of adenylate cyclase, intracellular cAMP levels rise. The breakdown of fat for fuel (lipolysis) is actually regulated by cAMP. Forskolin has been shown to not only enhance lipolysis but inhibits fat storage from occurring. This is appreciated by individuals trying to lose bodyfat obtain lean body mass. Another way that forskolin may allow for fat loss to occur is by stimulating thyroid hormone production and release. Thyroid hormone controls metabolism and can enhance metabolic rate, which may translate into more fat loss.

Generally, between about 50 milligrams and about 250 milligrams of a forskolin can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of forskolin. Generally, two, three, four or more dosages of the composition are taken over the course of a day to provide between about 50 milligrams and about 1000 milligrams of forskolin, e.g., between about 250 milligrams and about 500 milligrams.

Still another additive suitable for co-administration with the compositions of the invention is jojoba. Jojoba seed (Simmondsia chinensis), called Simmondsin, is a natural appetite suppressant.

Generally, between about 50 milligrams and about 250 milligrams of jojoba can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of jojoba. Generally, two, three, four or more dosages of the composition are taken over the course of a day to provide between about 50 milligrams and about 1000 milligrams of jojoba, e.g., between about 250 milligrams and about 500 milligrams.

Yet another additive suitable for co-administration with the compositions of the invention are guggul lipids. Gum Guggul (Commiphora mukul), also known as Guggul, Indian Bedellium, and Guggulow is a sticky gum resin from the mukul myrrh tree. Guggul has been found to lower cholesterol levels and also separately protected against the development of hardening of the arteries. The primary chemical constituents of Guggul include phytosterols, gugulipids, and guggulsterones. Guggul is also a weight loss agent that enhances thyroid function.

Generally, between about 50 milligrams and about 250 milligrams of a guggul can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of guggul. Generally, two, three, four or more dosages of the composition are taken over the course of a day to provide between about 50 milligrams and about 1000 milligrams of guggul, e.g., between about 250 milligrams and about 500 milligrams.

Other suitable additives for co-administration with the compositions of the invention include oleylethanolamide, N-oleoyl-phosphatidylethanolamine or amide and derivatives thereof that are cannabinoids useful for regulation of satiety and body weight. (See for example, Nature, 414, 209-212 (2001)). PHOSPHOLEAN™, a commercially available product from Chemi, S.p.A, Italy and Chemi Nutra, White Bear Lake, Minn., USA provides N-oleoyl-phosphatidyl ethanolamine, and is also known as NOPI.

Generally, between about 50 milligrams and about 250 milligrams of a cannabinoidscan be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of cannabinoid. Generally, two, three, four or more dosages of the composition are taken over the course of a day to provide between about 50 milligrams and about 1000 milligrams of cannabinoid, e.g., between about 250 milligrams and about 500 milligrams.

Still yet another additive suitable for co-administration with the compositions of the invention is Ephedra (Ephedra sinica). Also known as Ma Huang, (Ephedra) is a member of the family of herbs known as the Ephedracae. Ephedra contains two alkaloids, ephedrine and pseudoephedrine. Ephedra has been included in various weight loss and energy products. It helps to suppress the appetite and stimulates the thyroid gland that stimulates metabolism. Additionally, ma huang has been included in various supplements to treat obesity because of its thermogenic fat-burning effect on dietary intake.

Generally, between about 50 milligrams and about 250 milligrams of ephedra can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of ephedra. Generally, two, three, four or more dosages of the composition are taken over the course of a day to provide between about 50 milligrams and about 1000 milligrams of ephedra, e.g., between about 250 milligrams and about 500 milligrams.

Another additive suitable for co-administration with the compositions of the invention is yohimbe. Yohimbe is isolated from the inner bark of the tropical West African tree Corynanthe Yohimbe. Yohimbe helps to increase fatty acid mobilization and decreasing fat synthesis.

Generally, between about 50 milligrams and about 250 milligrams of yohimbe can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of yohimbe. Generally, two, three, four or more dosages of the composition are taken over the course of a day to provide between about 50 milligrams and about 1000 milligrams of yohimbe, e.g., between about 250 milligrams and about 500 milligrams.

Still another additive suitable for co-administration with the compositions of the invention is chromium picolinate. Chromium picolinate can lead to significant improvements in body composition resulting from fat loss, particularly for individuals who may not be as aggressive in making lifestyle changes such as reducing caloric intake or increasing their physical activity. It is believed that chromium picolinate's positive effect on body composition is through its ability to improve insulin utilization, thereby reducing fat deposition and resulting in improving entry of glucose and amino acids into muscle cells.

Generally, between about 50 milligrams and about 250 milligrams of a picolinate can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of picolinate. Generally, two, three, four or more dosages of the composition are taken over the course of a day to provide between about 50 milligrams and about 1000 milligrams of picolinate, e.g., between about 250 milligrams and about 500 milligrams.

Yet another additive suitable for co-administration with the compositions of the invention is garcinia cambodgia (commonly known as citrin or gambooge) that is rich in hydroxycitric acid (HCA), which is closely related to the citric acid found in grapefruits and oranges. HCA helps to promote weight loss in two basic ways. First, HCA blocks the conversion of sugary foods and starches into fats. Second, HCA is believed to raise levels of certain brain chemicals such as serotonin, a key regulator of appetite control. HCA also may suppress an individual's appetite.

Generally, between about 50 milligrams and about 250 milligrams of citrin can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of citrin. Generally, two, three, four or more dosages of the composition are taken over the course of a day to provide between about 50 milligrams and about 1000 milligrams of citrin, e.g., between about 250 milligrams and about 500 milligrams.

Still yet another suitable for co-administration with the compositions of the invention is fenugreek (Trigonella foenumgraecum). Fenugreek helps to regulate blood sugar regulation and/or glucose metabolism and helps stabilize normal sugar levels. It is believed that fenugreek also helps to increase the body's ability to lose stored body fat.

Generally, between about 50 milligrams and about 250 milligrams of fenugreek can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of fenugreek. Generally, two, three, four or more dosages of the composition are taken over the course of a day to provide between about 50 milligrams and about 1000 milligrams of fenugreek, e.g., between about 250 milligrams and about 500 milligrams.

Carnitine is a water-soluble vitamin like compound that the body utilizes to turn fat into energy. Carnitine works as part of an enzymatic complex formed from carnitine acyltransferase 1, camitine translocase and camitine transferase 11.

Carnitine is often used reduce cholesterol (LDL), increase high density lipoprotein (HDL), and for intermittent claudication. Although camitine does not increase blood flow, it is believe that it helps muscles to better function under difficult painful circumstances, such as those associated with claudication.

The actions of carnitine and CoQ-10 are interrelated. In fact, carnitine, through beta-oxidation of fatty acids, is able to restore the energy supplies necessary for cell-life, whereas Coenzyme Q is able to restore the ATP supplies necessary for the energetic metabolic processes of the cell.

L-carnitine is recognized in the art and facilitates transport of materials through the mitochondrial membrane. L-camitine is an essential fatty acid metabolism cofactor that helps to move fatty acids to the mitochondria from the cytoplasm. This is an important factor as this is where CoQ-10 uptake occurs.

The term “camitine” is also known as 3-Carboxy-2-hydroxy-N,N,N-trimethyl-1-propanaminium hydroxide, inner salt; (3-carboxy-2-hydroxypropyl)trimethylammonium hydroxide, inner salt; gamma-amino-beta-hydroxybutyric acid trimethylbetaine; gamma-trimethyl-beta-hydroxybutyrobetaine; 3-hydroxy-4-(trimethyl-ammonio)butanoate. See The Merck Index (1989), p. 281 and references cited therein. Therefore, “carnitine” and “carnitine analogs” includes, but is not limited to racemic or essentially pure L-carnitine (carnitine), or a corresponding alkanoyl-carnitine such as e.g. acetyl-carnitine or propionyl-carnitine, or a suitable salt of such compounds such as e.g. L-carnitine tartrate, L-carnitine fumarate, L-carnitine-magnesium-citrate, acetyl-L-carnitine tartrate, acetyl-L-carnitine-magnesium-citrate, or any mixture of the afore mentioned compounds.

Carnitine and carnitine analogs also include those described in U.S. Pat. Nos. 5,362,753, 4,687,782, 5,030,458, 5,030,657, 4,343,816, 5,560,928, 5,504,072, 5,391,550 and 5,240,961, the teachings of which are incorporated herein by reference in their entirety.

Generally, between about 50 milligrams and about 250 milligrams of carnitine or analog can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of carnitine or analog. Generally, two, three, four or more dosages of the composition are taken over the course of a day to provide between about 50 milligrams and about 1000 milligrams of carnitine, e.g., between about 250 milligrams and about 500 milligrams.

Panax ginseng is also called ginseng, Korean ginseng, schinsent, or ninjin. Ginseng is an adaptogen that has been used to lower cholesterol, balance the metabolism, increase energy levels, and stimulate the immune system.

Ginseng is characterized by the presence of ginsenoside. Ginsenosides are a class of steroid-like compounds, triterpene saponins, found exclusively in ginseng.

Generally, between about 25 milligrams and about 200 milligrams of ginseng is included in a composition of the invention, in particular, between about 50 milligrams and about 150 milligrams, and more particularly between about 75 milligrams and about 100 milligrams on a weight basis.

Typically a composition is provided that includes about 50 milligrams of ginseng. Generally, two, three, four or more dosages of the composition are taken over the course of a day to provide between about 25 and 200 milligrams of ginseng.

Cinnamon and its extracts can be included in the compositions of the invention for the aforementioned conditions. Typically the source for the extract is from a cinnamon tree, in the family of Cinnamomum. Species include Cinnamomum mairei, Cinnamomum zeylanicum, and Cinnamomum cassia. Commercial cinnamon bark which is the dried inner bark of the shoots and ground cinnamon obtained from food merchants can also be used for preparation of extracts. A commercially available source of cinnamon extract is Cinnulin PF™ (Integrity Nutraceuticals International, 201 Field End Street, Suite A, Sarasota, Fla. 34240) and is subject to U.S. Pat. No. 6,200,569.

Cinnamon is rich in antioxidant polyphenols, particularly procyanidin dimers and oligomers (OPCs). One of the polyphenols in cinnamon, known as methylhydroxy chalcone polymer, has been found to have particularly strong activity in the support of healthy blood sugar levels.

Generally, between about 50 milligrams and about 250 milligrams of cinnamon can be included in a composition of the invention, in particular, between about 75 milligrams and about 200 milligrams, and more particularly between about 100 milligrams and about 150 milligrams on a weight basis.

Typically a composition is provided that includes between about 50 milligrams and about 250 milligrams of cinnamon. Generally, two, three, four or more dosages of the composition are taken over the course of a day to provide between about 50 milligrams and about 1000 milligrams of the cinnamon, e.g., between about 250 milligrams and about 500 milligrams.

Coffee bean extracts, from both processed and green beans, provide plant phenols that include cinnamic acids, benzoic acids, flavonoids, proanthocyanidins, stilbenes, coumarins, lignans and lignins. These plant phenols have strong antioxidant activity. Important derivatives of cinnamic acids are chlorogenic acids.

Chlorogenic acids are a family of esters formed between trans-cinnamic acids and quinic acid. The most common chlorogenic acid is formed between caffeic acid and quinic acid. Both chlorogenic acid and caffeic acid are strong antioxidants. Chlorogenic acid is a phenolic natural product isolated from the leaves and fruits of dicotyledonous plants, including the coffee bean. Structurally, chlorogenic acid is the ester of caffeic acid with the 3-hydroxyl group of quinic acid.

Chlorogenic acid inhibits the hydrolysis of the glucose-6-phosphate enzyme in an irreversible fashion. Not to be limited by theory, this mechanism allows chlorogenic acid to reduce hepatic glycogenolysis (transformation of glycogen into glucose) and to reduce the absorption of new glucose. In addition, chlorogenic acid lessens the hyperglycemic peak resulting from the glycogenolysis brought about by the administering of glucagen, a hyperglycemiant hormone. Chlorogenic acid also assists in the reduction in blood glucose levels and an increase in the intrahepatic concentrations of glucose-6-phosphate and of glycogen.

Roasting of coffee beans dramatically increases their total antioxidant activity. Melanoidins are brown polymers formed by the Maillard reaction during the roasting of coffee beans. Melanoidins have significant antioxidant activity.

Generally, between about 50 milligrams and about 250 milligrams of a coffee bean extract or one or more of the constituents thereof can be included in a composition of the invention, in particular, between about 100 milligrams and about 200 milligrams, and more particularly between about 125 milligrams and about 175 milligrams on a weight basis.

Typically a composition is provided that includes between about 50 milligrams and about 250 milligrams of coffee bean extract or one or more of the constituents thereof. Generally, two, three, four or more dosages of the composition are taken over the course of a day to provide between about 50 milligrams and about 1000 milligrams of the additive(s), e.g., between about 250 and about 500 milligrams.

Chocolate extracts are also useful in the compositions of the invention, including polyphenols, pyrazines, quinoxalines, oxazolines, pyrroles (tannins), pyridines, flavonol proanthocyanidins, phenylethylamine, anandamide, methylxanthines, such as theobromine, theophylline and caffeine. Methylxanthines are thermogenic, meaning that the compound supports burning of calories to produce heat.

Caffeine, theophylline and theobromine inhibits the enzyme that breaks down cyclic adenosine monophosphate (cAMP), thus increasing availability of this high-energy compound that acts on receptors in many cells of the body, including fat and muscle cells. This is thought to be one of the primary mechanisms by which theobromine supports an increase in metabolic rate and the stimulation of fat breakdown (lipolysis).

Generally, between about 50 milligrams and about 250 milligrams of a chocolate extract or one or more of the constituents thereof can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of chocolate extract or one or more of the constituents thereof. Generally, two, three, four or more dosages of the composition are taken over the course of a day to provide between about 50 milligrams and about 1000 milligrams of the additive(s), e.g., between about 250 milligrams and about 500 milligrams.

Hoodia gordonii is another active ingredient that can be included in the compositions of the invention. It is a succulent plant from the botanical family Asclepiadaceae found in South Africa. Hoodia acts as an appetite suppressant and is useful for weight control. Hoodia contains variable amounts of fiber, organic material, antioxidants and biologically active substances, including steroidal glycosides, which appear to fool the brain into thinking the stomach is “full.” One such steroidal glycoside of importance is known as P57 or P57AS3 by Phytopharm PLC, Corpus Christi House, 9 West Street, Godmanchester, Cambridgeshire, United Kingdom (subject to a license to Pfizer, Inc. USA).

Generally, between about 50 milligrams and about 250 milligrams of hoodia gordonii can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of hoodia gordonii. Generally, two, three, four or more dosages of the composition are taken over the course of a day to provide between about 50 milligrams and about 1000 milligrams of the hoodia gordonii, e.g., between about 100 milligrams and about 500 milligrams.

Lotus root, lotus seed and extracts thereof provide asparaginic acid and vitamin B12. As used herein, the term “lotus leaf extract” refers to a solvent extract of lotus leaves (Nelumbo nucifera), such as an ethanol extract. The term also includes whole lotus leaves or seeds or any composition that includes a crude extract from lotus leaves. Lotus leaf extract is available commercially from, for instance, Advanced Herbal Ingredient Group, Inc., Changsha, China.

Generally, between about 50 milligrams and about 250 milligrams of lotus root, see or extract thereof can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of lotus seed, root or extract thereof. Generally, two, three, four or more dosages of the composition are taken over the course of a day to provide between about 100 milligrams and about 1000 milligrams of the additive(s).

It should be understood that the amounts of additives are based on a total composition weight of 1000 to 1500 milligrams per unit dose.

Compositions comprising the active compounds of the invention (or prodrugs thereof) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries that facilitate processing of the active compounds into preparations that can be used.

The active compound(s) or prodrug(s) thereof can be formulated in the pharmaceutical compositions per se, or in the form of a hydrate, solvate, or acceptable salt, as previously described. Typically, such salts are more soluble in aqueous solutions than the corresponding free acids and bases, but salts having lower solubility than the corresponding free acids and bases may also be formed.

The compositions of the invention may take a form suitable for virtually any mode of administration, including, for example, oral, buccal, systemic, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampoules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the compositions of the invention may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, cremophore™ or fractionated vegetable oils); and preservatives (e.g., methyl or propyl p hydroxybenzoates or sorbic acid). The preparations may also contain buffer salts, preservatives, flavoring, coloring and sweetening agents as appropriate.

Preparations for oral administration may be suitably formulated to give controlled release of the active compound or prodrug (esters and the like), as is well known.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For prolonged delivery, the active compound(s) or prodrug(s) can be formulated as a depot preparation for administration by implantation or intramuscular injection. The active ingredient may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the active compound(s) for percutaneous absorption, may be used. To this end, permeation enhancers may be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems may be employed. Liposomes and emulsions are well-known examples of delivery vehicles that may be used to deliver active compound(s) or prodrug(s). Certain organic solvents such as dimethylsulfoxide (DMSO) may also be employed, although usually at the cost of greater toxicity.

The compositions may, if desired, be presented in a pack or dispenser device, which may contain one or more unit dosage forms containing the active compound(s). The pack may, for example, comprise metal or plastic foil, such as a blister pack. The pack or dispenser device may be accompanied by instructions for administration.

Soft gel or soft gelatin capsules can be prepared, for example, without limitation, by dispersing the formulation in an appropriate vehicle (e.g., CLA, rice bran oil, and/or beeswax) to form a high viscosity mixture. This mixture is then encapsulated with a gelatin based film using technology and machinery known to those in the soft gel industry. The capsules so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is liquid (principally CLA, in combination with rice bran oil or wheat germ oil and/or beeswax if desired) and can include, apart form the antioxidant actives, a hydrophilic matrix. The hydrophilic matrix, if present, is a polyethylene glycol having an average molecular weight of from about 200 to 1000. Further ingredients are optionally thickening agents and/or emulsifying agent(s). In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerine, more particularly between about 17 and about 23 weight percent and more specifically about 20 weight percent glycerine.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams.

For example, a soft gel capsule can be prepared by mixing CLA, phaseolamin and GlucoTrim® as described throughout the specification. The mixture is then encapsulated within a soft gelatin capsule as described throughout.

Emulsifying agents that can be used to help solubilize the ingredients within the soft gelatin capsule include, for example, Specific examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

The present invention also provides packaged formulations of the compositions of the invention in a soft gel capsule and instructions for use of the product for weight related condition(s). Typically, the packaged formulation, in whatever form, is administered to an individual in need thereof that requires an increase in the amount of the composition in the individual's diet. Typically, the dosage requirement is between about 1 to about 4 dosages a day.

The phrase “reduce body fat” or “reduction of body fat” refers to a decrease in the amount of weight in an individual attributable to fat cells. Generally, this can be measured by many known methods, such as Body Mass Index, with skin fold calipers, by DEXA (Dual Energy X-ray Absorptiometry) and/or by hydrostatic weighing. It is intended that the present methods of the invention can reduce body fat by about 5%, more preferably by about 10% and most preferably about 20% or more of the total weight of the individual. Typically, this translates into a weight loss of about 2 to 3 pounds per week for an individual.

The phrase “increase glucose metabolism” is intended to mean that an individual's physiological ability to breakdown glucose is increased with a reduction in blood glucose levels.

The phrase “inhibit metabolism of carbohydrates” or “inhibition of metabolism of carbohydrates” is intended to mean that metabolism breakdown of carbohydrates into various constituents is prevented or decreased significantly. This is accomplished by one or more of the afore-mentioned starch blockers and is accomplished via differing mechanisms of action.

Although the present invention describes the preparation, use, manufacture and packaging of the compositions of the invention in soft gelatin capsules for treatment of various weight related conditions, it should not be considered limited to only soft gelatin capsules. Ingestible compositions of the invention can be delivered in traditional tablets, pills, lozenges, elixirs, emulsions, hard capsules, liquids, suspensions, etc. as described above.

The active compound(s) or prodrug(s) of the invention, or compositions thereof, will generally be used in an amount effective to achieve the intended result, for example in an amount effective to treat or prevent the particular weight related condition being treated. The composition may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a composition of the invention to a patient suffering from weight gain provides therapeutic benefit not only when the underlying condition is eradicated or ameliorated, but also when the patient reports a decrease in the severity or duration of the physical discomfort associated with the weight related condition.

For prophylactic administration, the composition may be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art.

Total dosage amounts will typically be in the range of from about 0.0001 or 0.001 or 0.01 mg/kg/day to about 100 mg/kg/day, but may be higher or lower, depending upon, among other factors, the activity of the components, its bioavailability, the mode of administration and various factors discussed above. Dosage amount and interval may be adjusted individually to provide plasma levels of the compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation.

In one aspect, the composition of the invention includes between about 5 milligrams (mg) and about 100 mg, more particularly between about 10 mg and about 50 mg, and between about 12 mg and about 25 mg of banaba extract per dosage, i.e., between about 10 mg and 30 mg. As previously discussed, the dosing can be administered by any number of delivery methods, i.e., soft gel capsules, tablets, in a foodstuff. Generally, the banaba extract provides about 3% corosolic acid based on the total weight of the banaba extract.

As a consequence, in one aspect, the composition of the invention include between about 0.06 mg and about 1.8 mg corosolic acid, more particularly between about 0.12 and about 1.5, and even more particularly between about 0.36 and about 0.48 mg.

In another aspect, the composition of the invention includes between about 100 mg and about 1000 mg of conjugated linoleic acid derivative, more particularly between about 200 mg and about 900 mg, and even more particularly between about 250 mg and about 750 mg, i.e., between about 500 mg and 750 mg.

In still another aspect, the composition of the invention includes between about 100 and about 1000 mg of starch blocker, more particularly between about 200 mg and about 800 mg, even more particularly between about 250 and about 750 mg, i.e., between about 500 mg and about 600 mg.

In one embodiment, a dosage per soft gel capsule would include between about 300 mg and about 800 mg conjugated linoleic acid derivative, i.e., between about 500 mg and 750 mg, between about 300 and about 800 mg starch blocker, i.e., Phaseolis vulgaris extract, i.e., between about 250 and 500 mg, and between about 5 mg and about 50 mg Banaba extract, i.e., between about 12 and 24 mg, providing about 0.15 to about 1.5 corosolic acid, based on a 3% Banaba extract.

Typically, an individual should administer a composition of the invention such that between about 1000 mg and about 3000 mg of CLA, between about 500 and about 1500 mg of starch blocker and between about 0.7 mg and about 2.2 mg of corosolic acid over a 24 hour period to achieve the desired effect(s) in the improvement of a weight related disorder(s). The composition can be administered in a single dose or in multiple doses.

In one embodiment, the gelatin used to prepare the soft gelatin capsule includes gelatin from lime or acid derived gel manufacturing processes known in the art. The gelatin is combined with plasticizers, such as glycerin, sorbitol or other polyalcoholic compounds, or combinations thereof and purified water. Optional additives can include colorants, preservatives, flavors, sweetening agents and/or opacifying agents. The amount of gelatin in the mixture can range from about 30 to about 60 percent (by weight), with about 15 to about 55% plasticizer (by weight) and purified water from about 15 to about 40% by weight. Optional additives are generally present in a range from about 0.1 to about 15% by weight.

A soft gel capsule would be prepared by mixing the conjugated linoleic acid derivative together with the starch blocker and banaba extract at a temperature between about 22 and about 30° C., for a period of time until the mixture was thoroughly mixed, optionally under vacuum. A gelatin mixture is fed into two spreader boxes, which in turn form two gelatin ribbons that are used to make each half of the gelatin capsule shell. The fill mixture (CLA, starch blocker and banaba extract as an example) is pumped into the gelatin ribbons held in place by two rotating die cavity rolls. The capsules are half sealed when a pump injects the fill material into the die cavities. The injection is followed by forming hermetic seals between the two capsule halves and the capsules are cut from the gelatin ribbon.

The capsules are dried at a temperature of a range of 70 to about 75° F. at a relative humidity of between about 15 and about 30 percent. Upon equilibration with the surrounding environment, the dried capsules will have a moisture content of between about 5 and about 10% by weight.

The following examples are intended to be illustrative only and should not be considered limiting.

### EXAMPLES

Formulations containing can be prepared in the following ratios by mixing the components together and then encapsulating into a soft gel capsule.

| Component | Example 1 | | Example 2 | |
|---|---|---|---|---|
| Starch Blocker | 250 | kg | 500 | kg |
| Banaba Extract (3% Corosolic acid) | 12 | kg | 24 | kg |
| CLA (60-87% by weight) | 500 | kg | 1000 | kg |
| Lecithin | 10 | kg | 25 | kg |
| Yellow Bee's wax | 20 | kg | 45 | kg |
| Total weight | 792 | kg | 1594 | kg |
| Component | Example 3 | | Example 4 | |
| Phaseolamin | 250 | kg | 500 | kg |
| Banaba Extract (3% Corosolic acid) | 12 | kg | 24 | kg |
| CLA (60-87% by weight) | 500 | kg | 1000 | kg |
| Lecithin | 10 | kg | 25 | kg |
| Yellow Bee's wax | 20 | kg | 45 | kg |
| Total weight | 792 | kg | 1594 | kg |
| Component | Example 5 | | Example 6 | |
| Starch Blocker | 250 | kg | 500 | kg |
| Banaba Extract (3% Corosolic acid) | 12 | kg | 24 | kg |
| CLA (60-87% by weight) | 500 | kg | 1000 | kg |
| Total weight | 762 | kg | 1524 | kg |
| Component | Example 7 | | Example 8 | |
| Phaseolamin | 250 | kg | 500 | kg |
| Banaba Extract (3% Corosolic acid) | 12 | kg | 24 | kg |
| CLA (60-87% by weight) | 500 | kg | 1000 | kg |
| Total weight | 762 | kg | 1524 | kg |

Total weight of fill material in each soft gelatin capsule was between about 792 mg and about 845 mg weight of a total capsule weight of about 1192 mg and about 1245 mg.

Although the present invention has been described with reference to preferred embodiments, persons skilled in the art will recognize that changes may be made in form and detail without departing from the spirit and scope of the invention.

All literature and patent references cited throughout the application are incorporated by reference into the application for all purposes.

What is claimed is:

1. A composition comprising a banaba extract, a conjugated linoleic acid derivative and a starch blocker.

2. The composition of claim 1, wherein said banaba extract is from Lagerstroemia Speciosa L.

3. The composition of claim 1, wherein said banaba extract comprises 3% corosolic acid by weight.

4. The composition of claim 1, wherein the conjugated linoleic acid derivative is conjugated linoleic acid, esters or salts thereof.

5. The composition of claim 1, wherein said starch blocker is an alpha amylase inhibitor.

6. The composition of claim 1, wherein said starch blocker is derived from Phaseolis vulgaris.

7. A method to reduce body fat, comprising the step of ingesting a composition comprising a banaba extract, a conjugated linoleic acid derivative and a starch blocker.

8. The method of claim 7, wherein said banaba extract is from Lagerstroemia Speciosa L.

9. The method of claim 7, wherein said banaba extract comprises 3% corosolic acid by weight.

10. The method of claim 7, wherein the conjugated linoleic acid derivative is conjugated linoleic acid, esters or salts thereof.

13. The method of claim 7, wherein said starch blocker is an alpha amylase inhibitor.

14. The method of claim 7, wherein said starch blocker is derived from Phaseolis vulgaris.

15. A packaged nutraceutical for reducing body fat, comprising a composition comprising a banaba extract, a conjugated linoleic acid derivative and a starch blocker; and
instructions for administering said fat reducing composition.

16. The packaged nutraceutical of claim 15, wherein said banaba extract is from Lagerstroemia Speciosa L.

17. The packaged nutraceutical of claim 15, wherein said banaba extract comprises 3% corosolic acid by weight.

18. The packaged nutraceutical of claim 15, wherein the conjugated linoleic acid derivative is conjugated linoleic acid, esters or salts thereof.

19. The packaged nutraceutical of claim 15, wherein said starch blocker is an alpha amylase inhibitor.

20. The packaged nutraceutical of claim 15, wherein said starch blocker is derived from Phaseolis vulgaris.

21. A method to increase glucose metabolism, comprising the step of ingesting a composition comprising a banaba extract, a conjugated linoleic acid derivative and a starch blocker.

22. The method of claim 21, wherein said banaba extract is from Lagerstroemia Speciosa L.

23. The method of claim 21, wherein said banaba extract comprises 3% corosolic acid by weight.

24. The method of claim 21, wherein the conjugated linoleic acid derivative is conjugated linoleic acid, esters or salts thereof.

25. The method of claim 21, wherein said starch blocker is an alpha amylase inhibitor.

26. The method of claim 21, wherein said starch blocker is derived from Phaseolis vulgaris.

27. A packaged nutraceutical for increasing glucose metabolism, comprising a composition comprising a banaba extract, a conjugated linoleic acid derivative and a starch blocker; and
instructions for administering said glucose metabolism increasing composition.

28. The packaged nutraceutical of claim 27, wherein said banaba extract is from Lagerstroemia Speciosa L.

29. The packaged nutraceutical of claim 27, wherein said banaba extract comprises 3% corosolic acid by weight.

30. The packaged nutraceutical of claim 27, wherein the conjugated linoleic acid derivative is conjugated linoleic acid, esters or salts thereof.

31. The packaged nutraceutical of claim 27, wherein said starch blocker is an alpha amylase inhibitor.

32. The packaged nutraceutical of claim 27, wherein said starch blocker is derived from Phaseolis vulgaris.

33. A method to inhibit metabolism of carbohydrates, comprising the step of ingesting a composition comprising a banaba extract, a conjugated linoleic acid derivative and a starch blocker.

34. The method of claim 33, wherein said banaba extract is from Lagerstroemia Speciosa L.

35. The method of claim 33, wherein said banaba extract comprises 3% corosolic acid by weight.

36. The method of claim 33, wherein the conjugated linoleic acid derivative is conjugated linoleic acid, esters or salts thereof.

37. The method of claim 33, wherein said starch blocker is an alpha amylase inhibitor.

38. The method of claim 33, wherein said starch blocker is derived from Phaseolis vulgaris.

39. A packaged nutraceutical for inhibiting metabolism of carbohydrates, comprising a composition comprising a banaba extract, a conjugated linoleic acid derivative and a starch blocker; and
instructions for administering said composition.

40. The packaged nutraceutical of claim 39, wherein said banaba extract is from Lagerstroemia Speciosa L.

41. The packaged nutraceutical of claim 39, wherein said banaba extract comprises 3% corosolic acid by weight.

42. The packaged nutraceutical of claim 39, wherein the conjugated linoleic acid derivative is conjugated linoleic acid, esters or salts thereof.

43. The packaged nutraceutical of claim 39, wherein said starch blocker is an alpha amylase inhibitor.

44. The packaged nutraceutical of claim 39, wherein said starch blocker is derived from Phaseolis vulgaris.

45. A method for weight loss, comprising the step of ingesting a composition comprising a banaba extract, a conjugated linoleic acid derivative and a starch blocker.

46. The method of claim 45, wherein said banaba extract is from Lagerstroemia Speciosa L.

47. The method of claim 45, wherein said banaba extract comprises 3% corosolic acid by weight.

48. The method of claim 45, wherein the conjugated linoleic acid derivative is conjugated linoleic acid, esters or salts thereof.

49. The method of claim 45, wherein said starch blocker is an alpha amylase inhibitor.

50. The method of claim 45, wherein said starch blocker is derived from Phaseolis vulgaris.

51. A packaged nutraceutical for weight loss, comprising a composition comprising a banaba extract, a conjugated linoleic acid derivative and a starch blocker; and
instructions for administering said composition.

52. The packaged nutraceutical of claim 52, wherein said banaba extract is from Lagerstroemia Speciosa L.

53. The packaged nutraceutical of claim 52, wherein said banaba extract comprises 3% corosolic acid by weight.

54. The packaged nutraceutical of claim 52, wherein the conjugated linoleic acid derivative is conjugated linoleic acid, esters or salts thereof.

55. The packaged nutraceutical of claim 52, wherein said starch blocker is an alpha amylase inhibitor.

56. The packaged nutraceutical of claim 52 wherein said starch blocker is derived from Phaseolis vulgaris.

57. A soft gel capsule comprising a composition of a banaba extract, a conjugated linoleic acid derivative and a starch blocker encapsulated within said soft gel capsule.

58. The soft gel capsule of claim 57, wherein said banaba extract is from Lagerstroemia Speciosa L.

59. The soft gel capsule of claim 57, wherein said banaba extract comprises 3% corosolic acid by weight.

60. The soft gel capsule of claim 57, wherein the conjugated linoleic acid derivative is conjugated linoleic acid, esters or salts thereof.

61. The soft gel capsule of claim 57, wherein said starch blocker is an alpha amylase inhibitor.

62. The soft gel capsule of claim 57, wherein said starch blocker is derived from Phaseolis vulgaris.
